(19) United States
(12) Patent Application Publication
(10) Pub. No.: US 2026/0108374 A1
(43) Pub. Date: Apr. 23, 2026

(54) NASAL STRIP FOR INCREASED AIR FLOW AND METHOD OF USING THE SAME

(71) Applicants: Wyatt Thurston, Phoenix, AZ (US); David Morelock, Atlanta, GA (US); Michael Morelock, Valley Center, CA (US)

(72) Inventors: Wyatt Thurston, Phoenix, AZ (US); David Morelock, Atlanta, GA (US); Michael Morelock, Valley Center, CA (US)

(21) Appl. No.: 19/365,038

(22) Filed: Oct. 21, 2025

Related U.S. Application Data
(60) Provisional application No. 63/710,203, filed on Oct. 22, 2024.

(51) Int. Cl. A61F 5/08

(52) U.S. Cl. CPC A61F 5/08

(57) ABSTRACT

Briefly, a nasal strip is provided that targets specific internal structures in the nose to efficiently and effectively open nasal passages for improved breathing and air flow. The nasal strip has (1) a vertical beam portion that may optionally be used to lift the end of the nose, (2) an upper lateral beam leaf spring that lifts the nose's internal upper cartilage, and (3) a lower lateral beam leaf spring that lifts the nose's internal lower cartilage. Each of these contributes to improved air flow in the nasal passageways. Further, the vertical beam portion acts as an attachment guide that facilitates repeatable and correct wearing of the nasal strip. In one construction, the nasal strip is a unitary body with three layers: a base adhesive layer, a middle leaf spring layer, and a covering layer.

### RELATED APPLICATION

This application claims priority to U.S. provisional application no. 63/710,203, filed Oct. 22, 2024, and entitled “Nose Strip for Increased Air Flow”, which is incorporated herein as if set forth in its entirety.

### FIELD OF THE INVENTION

The field of this invention is over-the-counter medical devices for improving nasal breathing and the resulting improved physical performance. More specifically, this invention relates to externally applied nasal strips that increase and ease air flow through the nose.

### BACKGROUND

Many people have restricted airflow through their nasal passages. This can be caused by various things, from genetics to allergies or even past injuries. Restricted airflow can be uncomfortable, distracting, and limit human performance. Several companies make nasal strips, such as Breath Right™, to relieve the blockage. All of the known strips on the market consist of a single laterally oriented beam that pulls the nostrils open. These products comprise a single lateral plastic leaf spring sandwiched between a skin adhesive layer and a cover layer.

These known nasal strips suffer from two serious deficiencies. First, it is difficult to consistently place them properly on the nose for efficient air flow. For example, they can be placed too high on the bridge of the nose, too close to the end of the nose, or can even be placed at an angle, with one side higher than the other such that the sides lack symmetry. Improper placement decreases the nasal strip's effectiveness and decreases wearability. Second, due to their size and orientation, known nasal strips have only a limited passage clearing effect, and tend to become uncomfortable even with limited wear.

Referring now to FIG. 1, an illustration of a nose 100 is shown with a known nasal strip 105 adhered to the nose 102. Nose 102 is shown in outline isometric, as well as views showing internal structure. When applied, the nasal strip 105 adheres only to the outer skin on nose 102 and does not directly touch the internal nasal structures. Nose 102 comprises several internal parts, but for the purposes of clarity of description, only the key parts will be discussed. Nose 102 has a nasal bone 107 that extends out from the skull from between the eyes and forms the general rigid shape for the upper part of the nose. A band of septal cartilage and bone 109 runs generally most of the length through the middle of the nasal passage separating the passage into two halves. The lower area of the nose 102 has a layer of two upper lateral cartilages 111, which for ease of description will often be referred to as the “upper cartilage” 111. A lower area of the nose 102 has two separate and distinct lower lateral cartilages 114, which will often be referred to as the “lower cartilage” 114. Finally, the lower sides of the nose 102 have alar fibrofatty tissue 117, which for ease of description will often be referred to as the “fatty tissue” 117. The upper and lower cartilages, supported by the septum, form the general shape of the external lower part of the nose. The upper and lower cartilages are flexible and support the general shape of the two internal nasal passages. As is well understood, these internal parts are covered by tissue and skin.

As illustrated, the nasal strip 105 is positioned adjacent to the upper cartilage 111. As such, the nasal strip 105 will be adhered to the skin, and its plastic leaf spring will act to pull the skin, tissue, and upper cartilage 111 up and away, which will cause the internal air passage in the nose 102 to open. When the nasal strip 105 is positioned over the upper cartilage 111, it will have very little effect on the lower cartilage 114, as these cartilage parts are separate and distinct. Positioning the nasal strip 105 adjacent the upper cartilage 111 is the preferred position.

However, if the user happens to place the nasal strip 105 further down the nose 102 at position 116, then the nasal strip 105 will be adjacent to the lower cartilage 114. As such, the nasal strip 105 will be adhered to the skin at position 116, and its plastic spring will act to pull the skin, tissue, and lower cartilage 114 up and away, which will cause the internal air passage in the nose 102 to open. When the nasal strip 105 is positioned over the lower cartilage 114 it will have very little effect on the upper cartilage 111, as these cartilage parts are separate and distinct. Positioning the nasal strip 105 adjacent the lower cartilage 114 near the end of the nose 102 is not a preferred position.

In some cases the user may place the nasal strip 105 at position 119, which is adjacent to both the upper cartilage 111 and the lower cartilage 114. When at position 119, the nasal strip 105 will have a limited opening effect on both the upper cartilage 111 and the lower cartilage 114. Also, since the nasal strip 105 has no positioning guide, the user may set the nasal strip 105 at an angle. In this way the nasal strip 105 would have a non-symmetrical opening effect on the nose 102. Finally, the known nasal strips 105 are unattractive and not aesthetically pleasing. As a result, the primary use for known nasal strips is in non-public places, for example, at home in bed as an anti-snoring aid.

What is needed is a new nasal strip that can provide a more repeatable and consistent opening effect for both the upper cartilage 111 and the lower cartilage 114. As well as being more functional, it would also be desirable to make the new nasal strips aesthetically pleasing to encourage public use.

### SUMMARY

An improved nasal strip is provided that targets specific internal structures in the nose to efficiently and effectively open nasal passages for improved breathing and air flow. The nasal strip has (1) a vertical beam portion that may optionally be used to lift the end of the nose, (2) an upper lateral beam leaf spring that lifts the nose's internal upper cartilage, and (3) a lower lateral beam leaf sprint that lifts the nose's internal lower cartilage. Each of these contributes to improved air flow in the nasal passageways. Further, the vertical beam portion acts as an attachment guide that facilitates repeatable and correct wearing of the nasal strip.

In one construction, the nasal strip is a unitary body with three layers: a base adhesive layer, a middle leaf spring layer, and a covering layer. The base layer typically has a protective paper sheet that a user removes prior to adhering the nasal strip to the nose. The leaf spring layer may be a plastic, such as a polyester film, although other plastics or memory metals may be used. The top layer is most often fabric, such as nylon or rayon, but other materials such as paper may be used.

Both the upper and lower lateral beam may be straight, curved, have a variable height, and extend orthogonally or at an angle relative to the vertical beam. The upper and lower lateral beam may be generally parallel to each other, or may diverge. The upper and lower lateral beam may even connect in some designs. With so many functional and aesthetic design options within the scope of this disclosure, the nasal strip can be constructed to be both functional and aesthetically pleasing.

### BRIEF DESCRIPTION OF THE DRAWINGS

FIG. 1 is an illustration of a prior art nasal strip in use on a nose.

FIGS. 2A-B are illustrations of nose anatomy.

FIGS. 3A-B are diagrams showing an embodiment of a nasal strip in accord with the present invention.

FIGS. 3A-C C is an illustration showing an embodiment of a nasal strip attached to a nose in accord with the present invention.

FIGS. 4A-B are diagrams showing an embodiment of a nasal strip in accord with the present invention.

FIG. 4C is an illustration showing an embodiment of a nasal strip attached to a nose in accord with the present invention.

FIGS. 5A-B are diagrams showing an embodiment of a nasal strip in accord with the present invention.

FIG. 5C is an illustration showing an embodiment of a nasal strip attached to a nose in accord with the present invention.

FIGS. 6A-B are diagrams showing an embodiment of a nasal strip in accord with the present invention.

FIG. 6C is an illustration showing an embodiment of a nasal strip attached to a nose in accord with the present invention.

FIG. 7 has illustrations showing embodiments of nasal strips in accord with the present invention.

FIG. 8 has illustrations showing embodiments of nasal strips in accord with the present invention.

FIG. 9 is a flowchart of using a nasal strip in accord with the present invention.

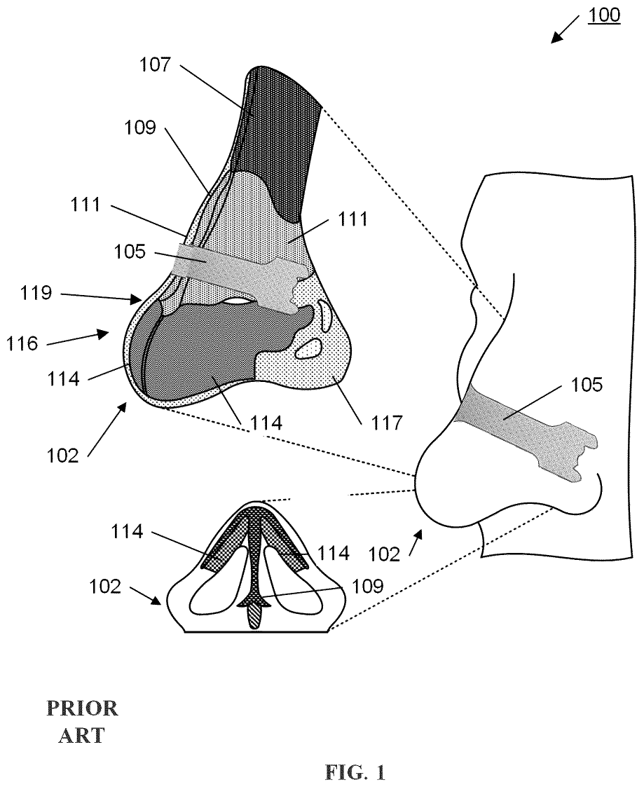

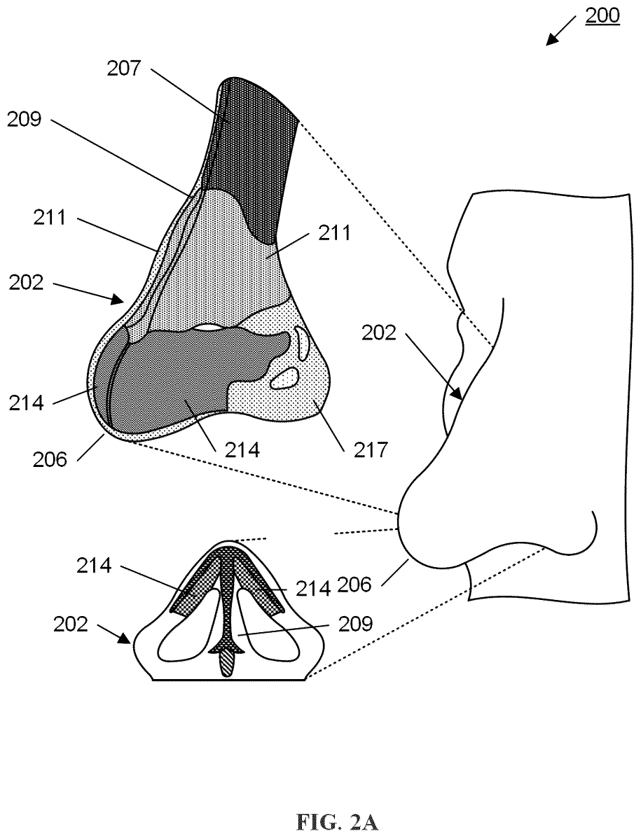

### DETAIL DESCRIPTION

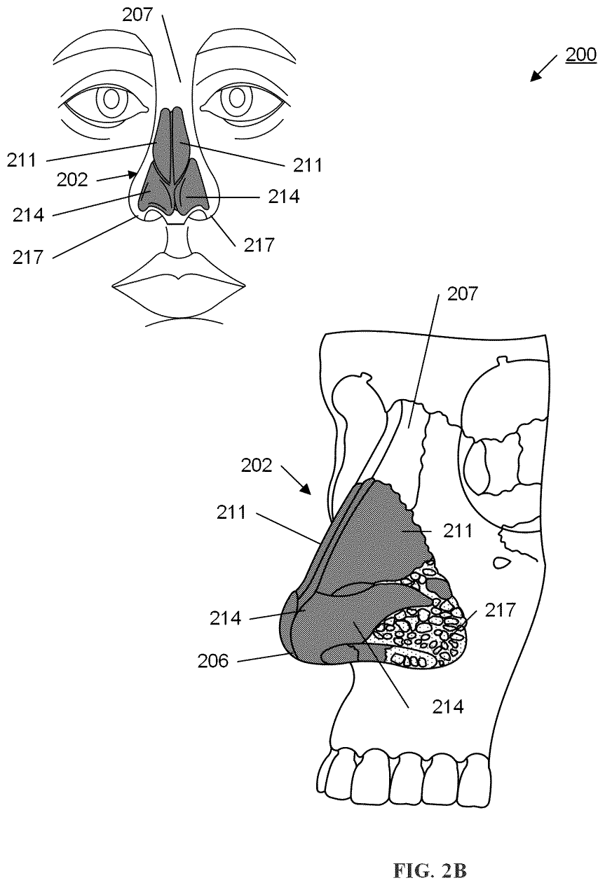

Referring now to FIG. 2A and FIG. 2B, further detail of the nose 200 is illustrated. Nose 202 has the nasal bone 207 extending away from the skull from a place generally between the eyes. The upper boney part of the nasal septum attaches along to the nasal bone 207. The lower cartilage part of the nasal septum 209 attaches to the upper and lower cartilages and extends to nearly the end 206 of the nose. Near the top of nose 202 is the upper cartilage 211, and nearer the end of the nose 202 is the lower cartilage 214. The upper cartilage 211 and the lower cartilage 214 have some overlap, but they are separate and distinct parts. Finally, near the outer lower edge of the nose 202 is the fatty tissue 217.

As a result of this nasal structure, the internal air passages of nose 202 can be opened in four ways: (1) pull the skin up and away at a position adjacent the upper cartilage 211; (2) pull the skin up and away at a position adjacent the lower cartilage 214; (3) pull skin up and away at a position adjacent the fatty tissue 217; or (4) pull the tip 206 of the nose upward and toward the skull. Each opening process has a positive effect on its own, but far more therapeutic effect is achieved by using multiple opening processes, which cannot be done using known nasal strips, such as nasal strip 105.

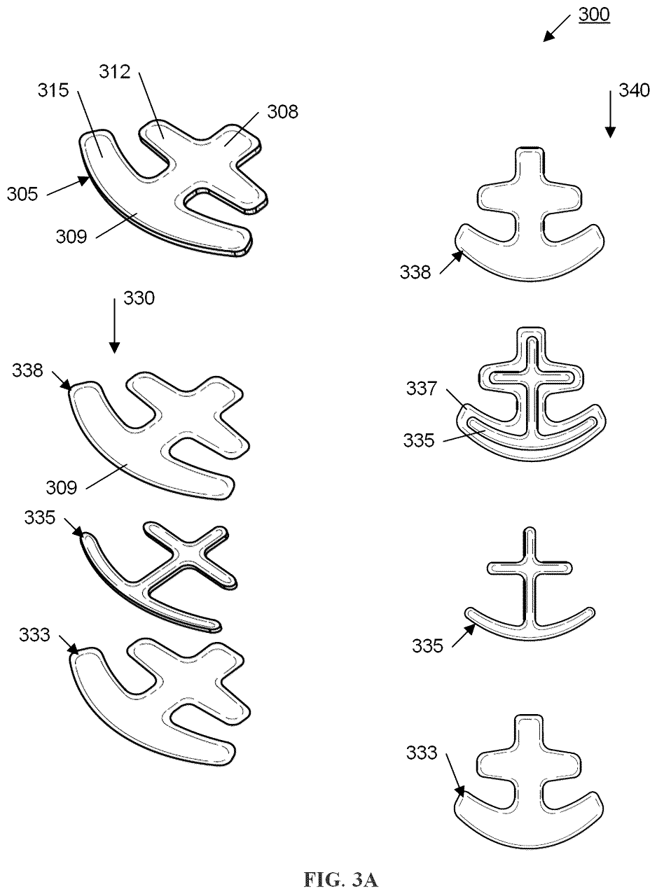

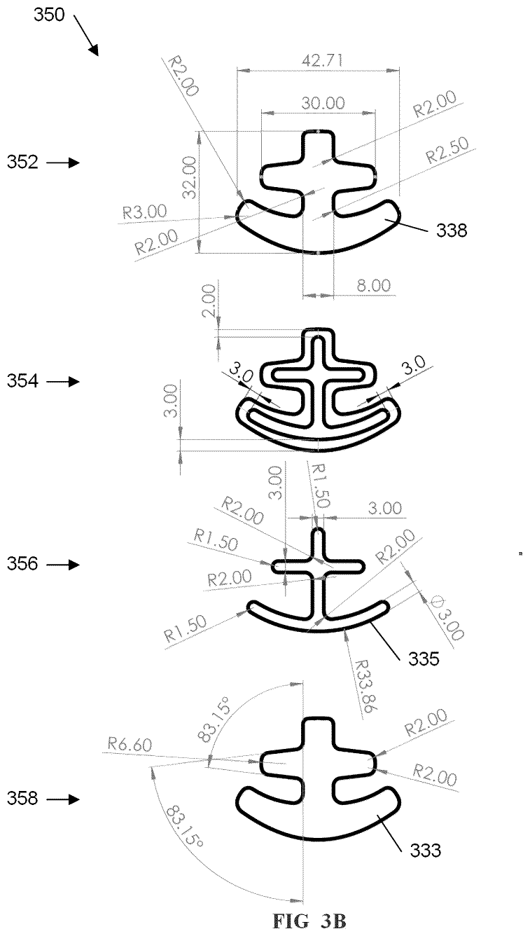

Referring now to FIGS. 2A-B and FIGS. 3A-C, an embodiment of an advanced nasal strip 300 is illustrated. Advantageously, nasal strip 305 can simultaneously (1) pull the skin up and away at a position adjacent the upper cartilage; (2) pull the skin up and away at a position adjacent the lower cartilage; (3) pull skin up and away at a position adjacent the fatty tissue; and (4) pull the tip of the nose upward and toward the skull. Further, nasal strip 305 has positioning guides that assist a user in consistently placing the nasal strip 305 in its optimum position.

Although nasal strip 305 is constructed as a unitary piece, it has three distinct functional portions. First, nasal strip 305 has a vertical beam portion 308, which extends the entire vertical length of the nasal strip 305. A user can adhere the distal end 309 of the vertical beam 308 at a position near the end of the nose 302 and pull back slightly as the vertical beam 308 is adhered along the top of the nose 302. Proper placement can thereby be consistent as the vertical beam 308 starts at a place near the end of the nose 302 and runs across the very top of the nasal bone. Further, adjusting how assertively the vertical beam is pulled back as it is being adhered will adjust how much the end of the nose 302 is pulled upward. In this way a user can pull less if improved comfort is desired or pull more aggressively if more air flow is desired. Pulling up the end of the nose will act to open the internal nasal air passages.

Second, the nasal strip 305 has an upper lateral beam portion 312. When the vertical beam 308 is properly placed as discussed above, then the upper lateral beam portion 312 is properly positioned adjacent to the upper cartilage 211 and is symmetrical. As such, when the upper lateral beam 312 is adhered to the nose 302, it acts to pull the upper cartilage 211 up and away, which will open the internal nasal air passages. The upper lateral beam portion 312 may be generally positioned horizontally in relation to the vertical beam. However, it will be appreciated that the upper lateral beam portion 312 may be straight, curved or take on other geometric shapes. However, the upper lateral beam portion 312 will be designed and shaped to primarily pull the upper cartilage 211 up and away so as to open internal nasal air passages.

Third, the nasal strip 305 has a lower lateral beam portion 315. When the vertical beam 308 is properly placed as discussed above, then the lower lateral beam portion 315 is properly positioned adjacent to the lower cartilage 214 and is symmetrical. As such, when the lower lateral beam 315 is adhered to the nose 302, it acts to pull the lower cartilage 214 up and away, which will open the internal nasal air passages. The lower lateral beam 315 further acts to pull fatty tissue 217 up and away, which also acts to open the internal nasal air passages. The lower lateral beam portion 315 may be generally positioned horizontally in relation to the vertical beam. However, it will be appreciated that the lower lateral beam portion 315 may be straight, curved or take on other geometric shapes. However, the lower lateral beam portion 315 will be designed and shaped to primarily pull the lower cartilage 214 up and away so as to open internal nasal air passages.

Additionally the lower lateral beam portion 315 may be longer than the upper lateral beam portion 312, or may be constructed in a different lengths and geometric shape.

As illustrated in exploded view 330, the nasal strip comprises 3 layers. A bottom adhesive layer 333 is provided, which has a removable paper protector (not shown). When the protective layer is removed by the user, then the adhesive layer 333 removably attaches to the skin of the nose 302. A middle layer 335 is a semi-rigid plastic material such as polyester. In this way, the lateral beam portions (312, 315) function as springs, which can then provide the upward and outward force to open the air passages. It will be understood that there are several plastic and metal alternatives that can function as the leaf spring middle layer 335.

Finally, a top layer 338 is provided, for example, a colored fabric material. In one embodiment, the top layer 338 is a dyed rayon material. It will be understood that many fabric and non-fabric materials may function as the top layer 338. As illustrated in exploded top view 340, The polyester middle layer 335 is set on a medical grade adhesive tape to facilitate assembly.

More detailed assembly guides 350 illustrate one specific embodiment of the nasal strip 308. View 352 shows the dimensions of top layer 338. In one example top layer 338 is a colored rayon fabric. View 356 shows middle layer 335, which is one embodiment of biocompatible polyester film that is 0.3 mm thick. It will be understood that other plastics or metals may be used, and that thickness can be adjusted to provide the desired leaf spring effect. View 354 shows the middle polyester layer 335 placed on an adhesive medical tape. In this way, the middle layer 335 is coupled to the top layer 338. View 358 shows the bottom layer 333, which in one embodiment can be a hypoallergenic non-woven medial tape that is biocompatible. Ideally this will be a breathable adhesive material that adheres to skin. Such material can adhere immediately upon contact with the skin and should remain adhered and comfortable for at least 4-6 hours. In some cases, such as when asleep or less active, longer wear times may be achieved.

Illustration 375 shows the nasal strip 305 adhered to a user. The vertical beam portion 308 extends from the tip of the nose 302 and runs along the top of the nose toward the skull. As such, the vertical beam 308 acts as a repeatable and consistent positioning aid for the nasal strip 308. Also, the vertical beam 308 can be pulled back during application to adjust how aggressively the end of the nose is lifted. Lifting the end of the nose acts to open the internal nasal air passages. The upper lateral beam 312 is positioned adjacent to the upper cartilage, and when adhered to the side of the nose, acts to pull the skin and the upper cartilage up and away, which acts to open the internal air passageways. The lower lateral beam 315 is positioned adjacent to the lower cartilage, and when adhered to the side of the nose, acts to pull the skin and the lower cartilage up and away, which acts to open the internal air passageways. Further, the lower lateral beam will act to pull the fatty tissue 317 up and away, which also acts to open the internal nasal air passageways.

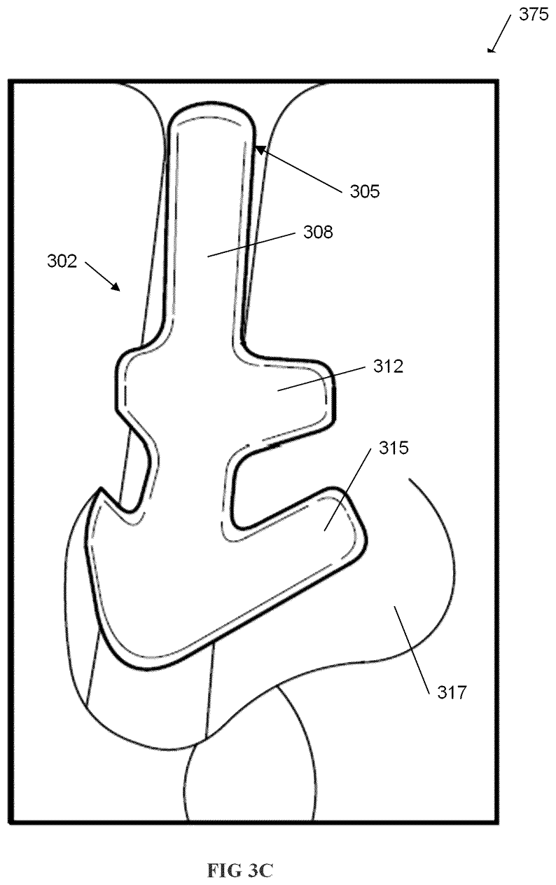

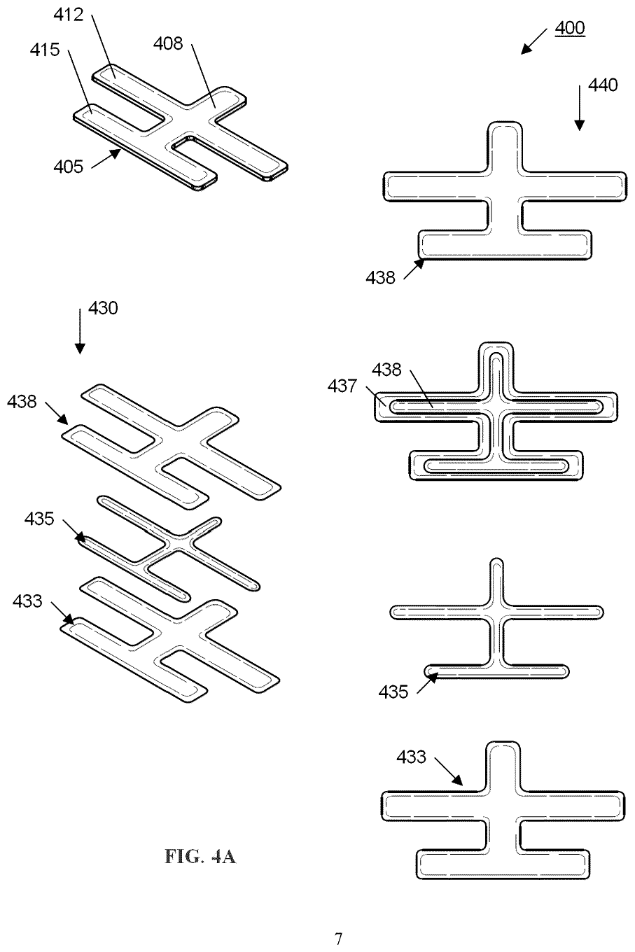

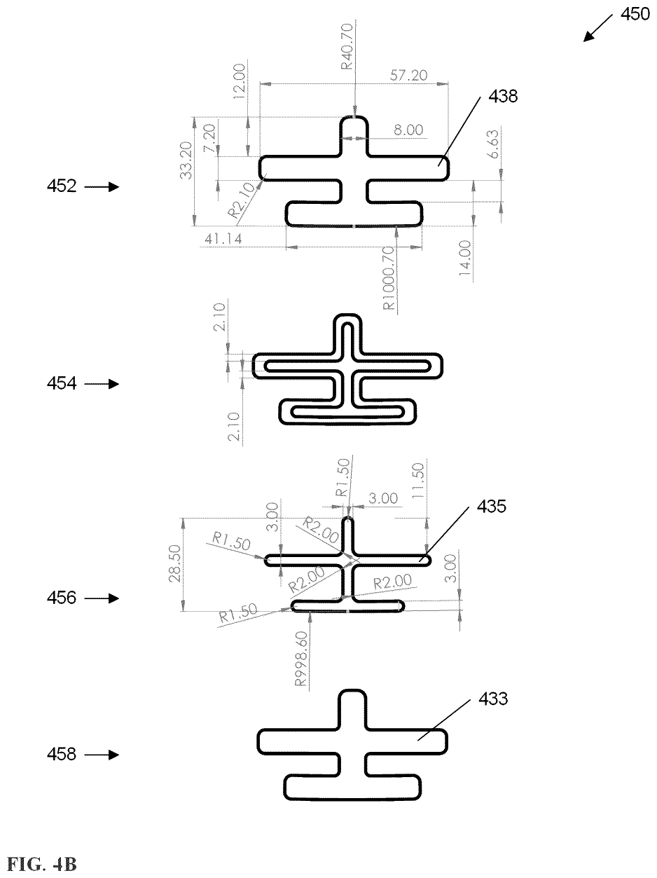

Referring now to FIGS. 2A-B and FIGS. 4A-C, an embodiment of an advanced nasal strip 400 is illustrated. Advantageously, nasal strip 405 can simultaneously (1) pull the skin up and away at a position adjacent the upper cartilage; (2) pull the skin up and away at a position adjacent the lower cartilage; (3) pull skin up and away at a position adjacent the fatty tissue; and (4) pull the tip of the nose upward and toward the skull. Further, nasal strip 405 has positioning guides that assist a user in consistently placing the nasal strip 405 in its optimum position.

Nasal strip 400 is similar to nasal strip 300 discussed with reference to FIGS. 3A-C. Accordingly, nasal strip 400 will not be described in detail, but incorporates the relevant descriptions from FIGS. 3A-C. The upper lateral beam 412 and lower lateral beam 415 are positioned orthogonally to the vertical beam portion 408. They have similar heights, but the upper lateral beam 412 is longer. It will be understood that many other dimensions, angles, and geometries may be used consistent with this disclosure.

Although nasal strip 405 is constructed as a unitary piece, it has three distinct functional portions. First, nasal strip 405 has a vertical beam portion 408, which extends the entire vertical length of the nasal strip 405. Proper placement can be consistent as the vertical beam 408 starts at a place near the end of the nose 402, and runs across the very top of the nasal bone. Further, adjusting how assertively the vertical beam 408 is pulled back as it is being adhered will adjust how much the end of the nose 402 is pulled upward. Second, the nasal strip 405 has an upper lateral beam portion 412. When the vertical beam 408 is properly placed as discussed above, then the upper lateral beam portion 412 is properly positioned adjacent to the upper cartilage 211 and is symmetrical. Third, the nasal strip 305 has a lower lateral beam portion 415. When the vertical beam 408 is properly placed as discussed above, then the lower lateral beam portion 415 is properly positioned adjacent to the lower cartilage 214 and is symmetrical.

As illustrated in exploded view 430, the nasal strip comprises 3 layers. A bottom adhesive layer 433 is provided, which has a removable paper protector (not shown). A middle layer 4335 is a semi-rigid plastic material such as polyester. In this way, the lateral beam portions (412, 415) function as leaf springs, which can then provide the upward and outward force to open the air passages. Finally, a top layer 438 is provided, for example, a colored fabric material. In one embodiment, the top layer 438 is a dyed rayon material. Additional relevant detail description is made with reference to FIGS. 3A-C, but for ease of explanation will not be repeated here.

More detailed assembly guides 450 illustrate one specific embodiment of the nasal strip 408. View 452 shows the dimensions of top layer 438. View 456 shows middle layer 435, which is one embodiment of biocompatible PET film that is 7 mm thick. View 454 shows the middle polyester layer 435 placed on an adhesive medical tape. In this way, the middle layer 435 is coupled to the top layer 438. View 458 shows the bottom layer 433, which in one embodiment can be a hypoallergenic non-woven medial tape that is biocompatible. Additional relevant detail description is made with reference to FIGS. 3A-C, but for ease of explanation will not be repeated here.

Illustration 475 shows the nasal strip 405 adhered to a user. The vertical beam portion 408 extends from the tip of the nose 402 and runs along the top of the nose toward the skull. As such, the vertical beam 408 acts as a repeatable and consistent positioning aid for the nasal strip 408. Also, the vertical beam 408 can be pulled back during application to adjust how aggressively the end of the nose is lifted. Lifting the end of the nose acts to open the internal nasal air passages. The upper lateral beam 412 is positioned adjacent to the upper cartilage, and when adhered to the side of the nose, acts to pull the skin and the upper cartilage up and away, which acts to open the internal air passageways. The lower lateral beam 415 is positioned adjacent to the lower cartilage, and when adhered to the side of the nose, acts to pull the skin and the lower cartilage up and away, which acts to open the internal air passageways. Further, the lower lateral beam will act to pull the fatty tissue 417 up and away, which also acts to open the internal nasal air passageways.

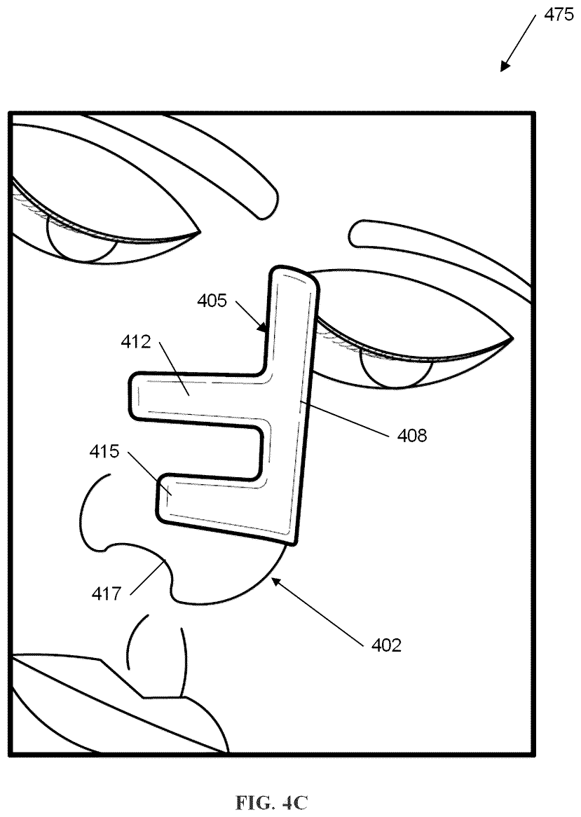

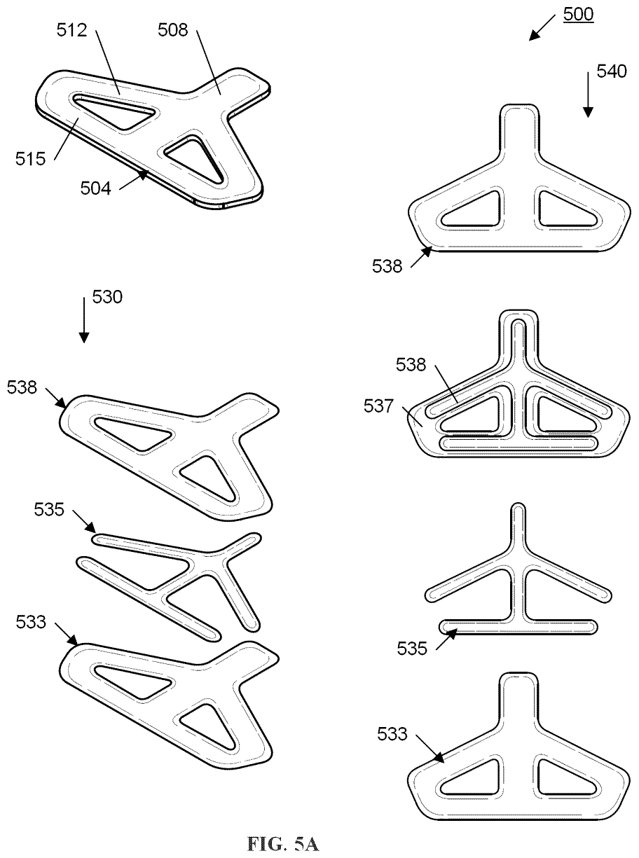

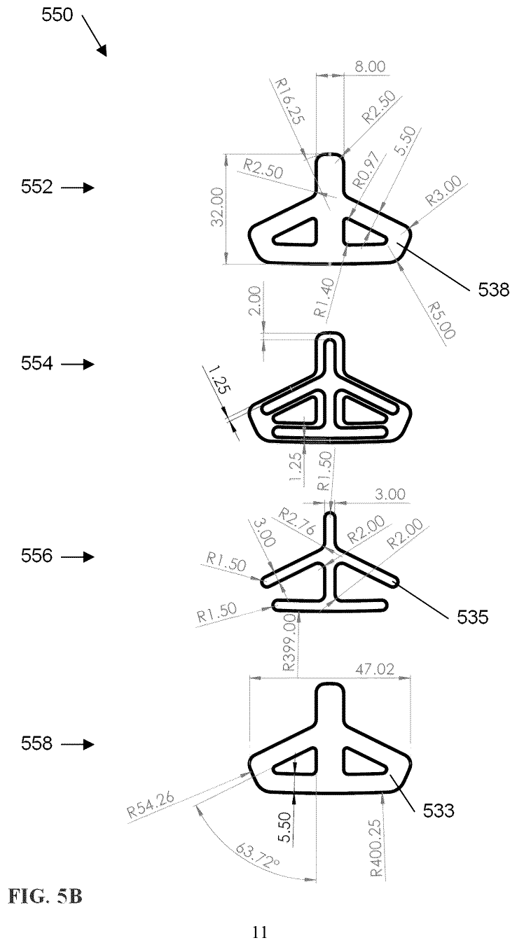

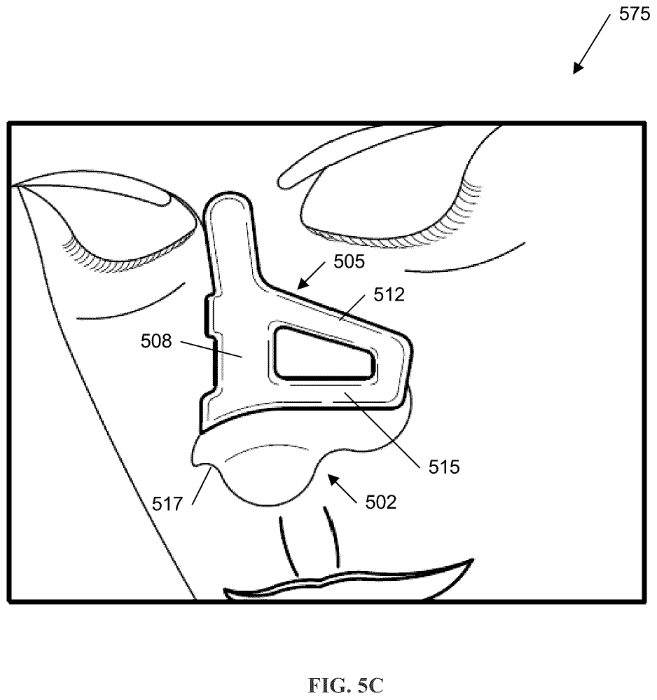

Referring now to FIGS. 2A-B and FIGS. 5A-C, an embodiment of an advanced nasal strip 500 is illustrated. Advantageously, nasal strip 505 can simultaneously (1) pull the skin up and away at a position adjacent the upper cartilage; (2) pull the skin up and away at a position adjacent the lower cartilage; (3) pull skin up and away at a position adjacent the fatty tissue; and (4) pull the tip of the nose upward and toward the skull. Further, nasal strip 505 has positioning guides that assist a user in consistently placing the nasal strip 505 in its optimum position.

Nasal strip 500 is similar to nasal strip 300 discussed with reference to FIGS. 3A-C. Accordingly, nasal strip 500 will not be described in detail, but incorporates the relevant descriptions from FIGS. 3A-C. The upper lateral beam 512 is angled downward in relation to the vertical beam portion 508. The lower lateral beam 515 is at the base of the vertical beam portion 508 and is positioned orthogonally to the vertical beam portion 508. The bottom 533 and top layer 539 provide a limited connection between the upper lateral beam 512 and the lower lateral beam 515, but this connection is primarily for aesthetic purposes. On the middle leaf spring, the upper lateral beam 512 and the lower lateral beam 515 are not connected at their distal ends. In an alternative embodiment, the upper lateral beam 512 and the lower lateral beam 515 could be connected at their distal ends Even if connected, the upper lateral beam 512 primarily pulls on the upper cartilage 211 and lower lateral beam 515 primarily pulls on the lower cartilage 214 and fatty tissue 217. It will be understood that many other dimensions and geometries may be used consistent with this disclosure.

Although nasal strip 505 is constructed as a unitary piece, it has three distinct functional portions. First, nasal strip 505 has a vertical beam portion 508, which extends the entire vertical length of the nasal strip 505. Proper placement can be consistent as the vertical beam 508 starts at a place near the end of the nose 502 and runs across the very top of the nasal bone. Further, adjusting how assertively the vertical beam 508 is pulled back as it is being adhered will adjust how aggressively the end of the nose 502 is pulled upward. Second, the nasal strip 505 has an upper lateral beam portion 512. When the vertical beam 508 is properly placed as discussed above, then the upper lateral beam portion 512 is properly positioned adjacent to the upper cartilage 511 and is symmetrical. Third, the nasal strip 505 has a lower lateral beam portion 515. When the vertical beam 508 is properly placed as discussed above, then the lower lateral beam portion 515 is properly positioned adjacent to the lower cartilage 514 and is symmetrical.

As illustrated in exploded view 530, the nasal strip comprises 3 layers. A bottom adhesive layer 533 is provided, which has a removable paper protector (not shown). A middle layer 535 is a semi-rigid plastic material such as polyester. In this way, the lateral beam portions (512, 515) function as springs, which can then provide the upward and outward force to open the air passages. Finally, a top layer 538 is provided, for example, a colored fabric material. In one embodiment, the top layer 538 is a dyed rayon material. Additional relevant detail description is made with reference to FIGS. 3A-C, but for ease of explanation will not be repeated here.

More detailed assembly guides 550 illustrate one specific embodiment of the nasal strip 508. View 552 shows the dimensions of top layer 538. View 556 shows middle layer 535, which is one embodiment of biocompatible polyester film that is 0.3 mm thick. View 554 shows the middle polyester layer 535 placed on an adhesive medical tape. In this way, the middle layer 535 is coupled to the top layer 538. View 558 shows the bottom layer 533, which in one embodiment can be a hypoallergenic non-woven medial tape that is biocompatible. Additional relevant detail description is made with reference to FIGS. 3A-C, but for ease of explanation will not be repeated here.

Illustration 575 shows the nasal strip 505 adhered to a user. The vertical beam portion 508 extends from the tip of the nose and runs along the top of the nose toward the skull. As such, the vertical beam 508 acts as a repeatable and consistent positioning aid for the nasal strip 508. Also, the vertical beam 508 can be pulled back during application to adjust how much the end of the nose is lifted. Lifting the end of the nose acts to open the internal nasal air passages. The upper lateral beam 512 is positioned adjacent to the upper cartilage, and when adhered to the side of the nose, acts to pull the skin and the upper cartilage up and away, which acts to open the internal air passageways. The lower lateral beam 515 is positioned adjacent to the lower cartilage, and when adhered to the side of the nose, acts to pull the skin and the lower cartilage up and away, which acts to open the internal air passageways. Further, the lower lateral beam will act to pull the fatty tissue 517 up and away, which also acts to open the internal nasal air passageways.

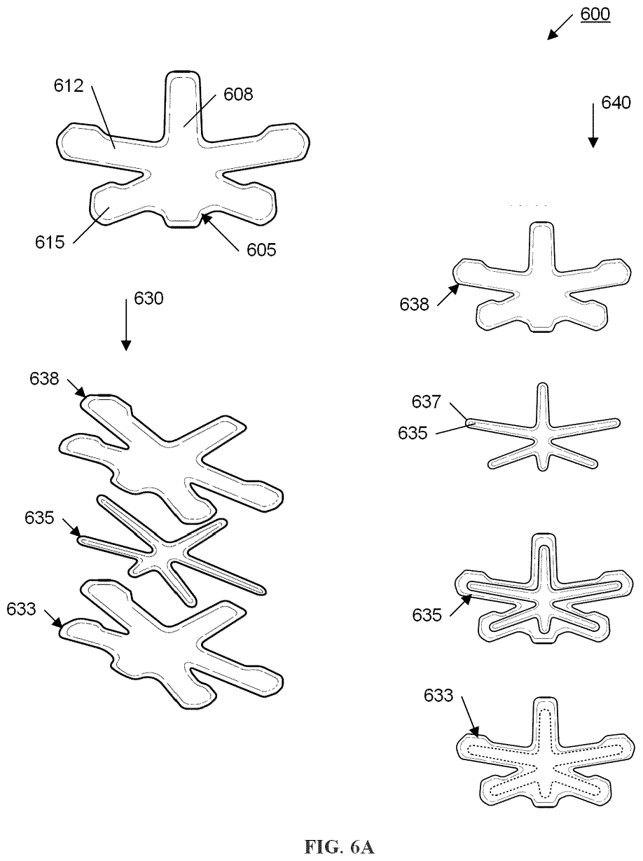

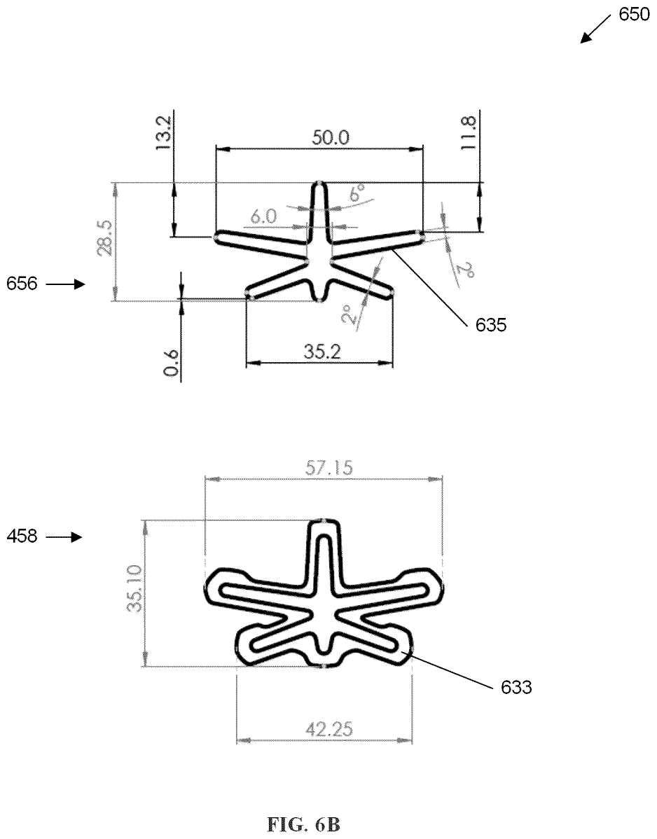

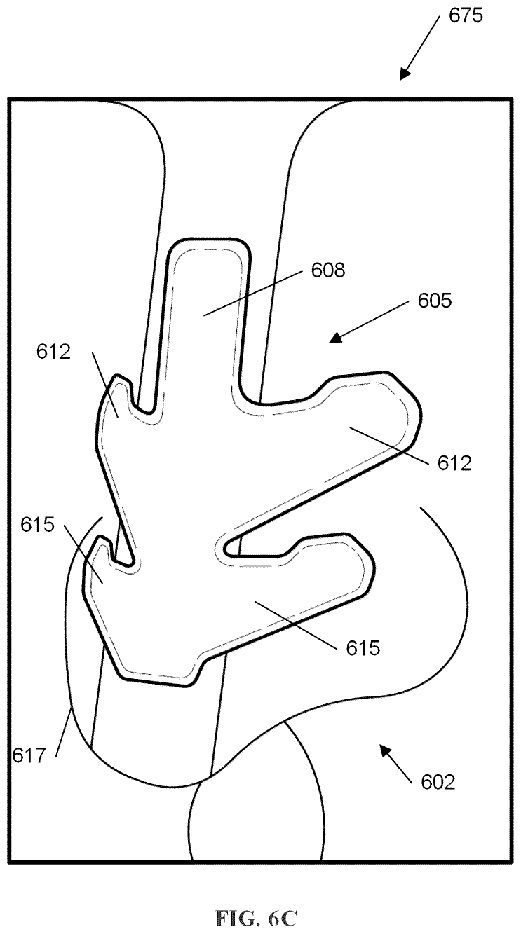

Referring now to FIGS. 2A-B and FIGS. 6A-C, an embodiment of an advanced nasal strip 600 is illustrated. Advantageously, nasal strip 605 can simultaneously (1) pull the skin up and away at a position adjacent the upper cartilage; (2) pull the skin up and away at a position adjacent the lower cartilage; (3) pull skin up and away at a position adjacent the fatty tissue; and (4) pull the tip of the nose upward and toward the skull. Further, nasal strip 605 has positioning guides that assist a user in consistently placing the nasal strip 605 in its optimum position.

Nasal strip 600 is similar to nasal strip 300 discussed with reference to FIGS. 3A-C. Accordingly, nasal strip 600 will not be described in detail, but incorporates the relevant descriptions from FIGS. 3A-C. The upper lateral beam 612 extends at a slight upward angle from the vertical beam 608, while the lower lateral beam 615 extends at a slight downward angle from the vertical beam portion 608. They have similar heights, but the upper lateral beam 612 is longer. Each distal end of the lateral beams (612, 615) has an enlarged portion. It will be understood that many other dimensions, lengths and geometries may be used consistent with this disclosure.

Although nasal strip 605 is constructed as a unitary piece, it has three distinct functional portions. First, nasal strip 605 has a vertical beam portion 608, which extends the entire vertical length of the nasal strip 605. Proper placement can be consistent as the vertical beam 608 starts at a place near the end of the nose 602 and runs across the very top of the nasal bone. Further, adjusting how assertively the vertical beam 608 is pulled back as it is being adhered will adjust how much the end of the nose 602 is pulled upward. Second, the nasal strip 605 has an upper lateral beam portion 612. When the vertical beam 608 is properly placed as discussed above, then the upper lateral beam portion 612 is properly positioned adjacent to the upper cartilage 211 and is symmetrical. Third, the nasal strip 605 has a lower lateral beam portion 615. When the vertical beam 608 is properly placed as discussed above, then the lower lateral beam portion 415 is properly positioned adjacent to the lower cartilage 214 and is symmetrical.

As illustrated in exploded view 630, the nasal strip comprises 3 layers. A bottom adhesive layer 633 is provided, which has a removable paper protector (not shown). A middle layer 635 is a semi-rigid plastic material such as polyester. In this way, the lateral beam portions (612, 615) function as leaf springs, which can then provide the upward and outward force to open the air passages. Finally, a top layer 638 is provided, for example, a colored fabric material. In one embodiment, the top layer 638 is a dyed rayon material. Additional relevant detail description is made with reference to FIGS. 3A-C, but for ease of explanation will not be repeated here.

More detailed assembly guides 650 illustrate one specific embodiment of the nasal strip 608. View 656 shows middle layer 635, which is one embodiment of biocompatible PET film that is 7 mm thick. The middle polyester layer 635 is placed on an adhesive medical tape (not shown). In this way, the middle layer 635 is coupled to the top layer 638. View 658 shows the bottom layer 633, which in one embodiment can be a hypoallergenic non-woven medial tape that is biocompatible. Additional relevant detail description is made with reference to FIGS. 3A-C, but for ease of explanation will not be repeated here.

Illustration 675 shows the nasal strip 505 adhered to a user. The vertical beam portion 608 extends from the tip of the nose and runs along the top of the nose toward the skull. As such, the vertical beam 608 acts as a repeatable and consistent positioning aid for the nasal strip 608. Also, the vertical beam 608 can be pulled back during application to adjust how much the end of the nose is lifted. Lifting the end of the nose acts to open the internal nasal air passages. The upper lateral beam 612 is positioned adjacent to the upper cartilage, and when adhered to the side of the nose, acts to pull the skin and the upper cartilage up and away, which acts to open the internal air passageways. The lower lateral beam 615 is positioned adjacent to the lower cartilage, and when adhered to the side of the nose, acts to pull the skin and the lower cartilage up and away, which acts to open the internal air passageways. Further, the lower lateral beam will act to pull the fatty tissue 617 up and away, which also acts to open the internal nasal air passageways.

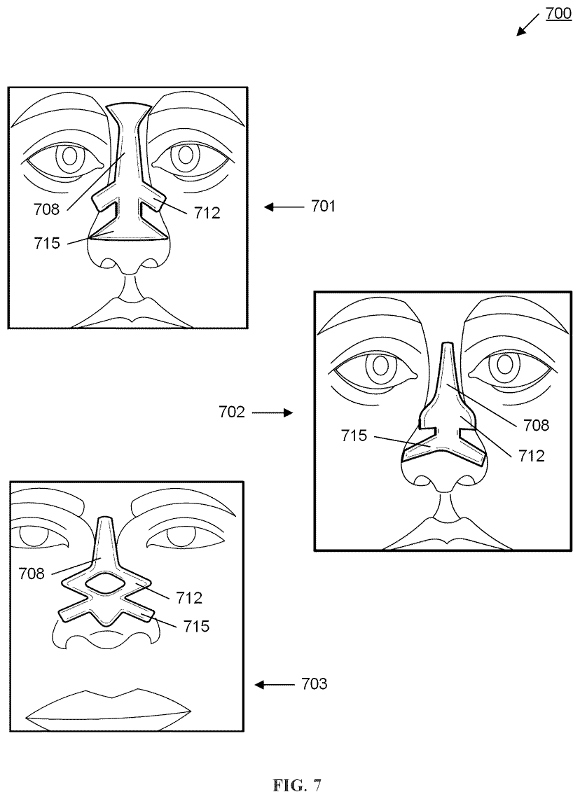

FIG. 7 has three additional designs 700 for advanced nasal strips. Each of the designs shown in FIG. 7 can simultaneously (1) pull the skin up and away at a position adjacent the upper cartilage; (2) pull the skin up and away at a position adjacent the lower cartilage; (3) pull skin up and away at a position adjacent the fatty tissue; and (4) pull the tip of the nose upward and toward the skull. Further, each nasal strip in FIG. 7 has positioning guides that assist a user in consistently placing the nasal strip 705 in its optimum position. The designs in FIG. 7 are similar to the designs discussed with reference to previously discussed designs, so will not be described in detail.

View 701 shows an advanced nasal strip having a vertical beam 708, an upper lateral beam 712 and a lower lateral beam 715. In this design, the upper portion of the vertical beam is generally inverted-triangular shaped and then widens as it extends down the nose. The upper lateral beam 712 is angled downward in relation to the vertical beam portion 708 and has the same height as it extends outward. The lower lateral beam 715 is at the base of the vertical beam portion 708 and is positioned orthogonally to the vertical beam portion 708. The lower lateral beam 715 is wider near the vertical beam 708 and narrows to a point at its distal end. It will be understood that many other dimensions, angles, and geometries may be used consistent with this disclosure.

View 702 shows an advanced nasal strip having a vertical beam 708, an upper lateral beam 712 and a lower lateral beam 715. In this design, the vertical beam 708 has a similar width for the full length of the nose. The upper lateral beam 712 is generally orthogonal in relation to the vertical beam portion 708 and its height decreases as it extends from the vertical beam 708. The lower lateral beam 715 is at the base of the vertical beam portion 708 and is positioned orthogonally to the vertical beam portion 708. The lower lateral beam 715 is wider near the vertical beam 708 and narrows somewhat as it extends away from the vertical beam 708. In this design the lower lateral beam 715 is longer than the upper lateral beam 712. It will be understood that many other dimensions, angles, and geometries may be used consistent with this disclosure.

View 703 shows an advanced nasal strip having a vertical beam 708, an upper lateral beam 712 and a lower lateral beam 715. In this design, the vertical beam 708 has a void midspan, but tension is maintained as the upper lateral beam 712 and the lower lateral beam 715 are operably coupled. The upper lateral beam 712 is first angled downward in relation to the vertical beam portion 708 and then forms a “V” angle to reconnect with the vertical beam portion 708. Its height remains generally constant for its full length. The lower lateral beam 715 is angled downward in relation to the vertical beam portion 708, and has a generally constant height throughout its length. In this design the upper lateral beam 715 is longer than the lower lateral beam 712, primarily due to its “V” shaped angle. It will be understood that many other dimensions and geometries may be used consistent with this disclosure.

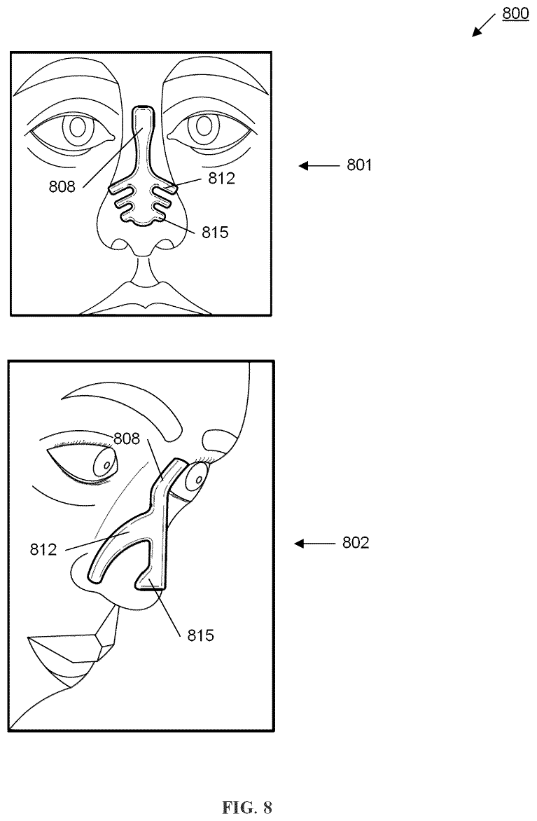

FIG. 8 has two additional designs 800 for advanced nasal strips. Each of the designs shown in FIG. 8 can simultaneously (1) pull the skin up and away at a position adjacent the upper cartilage; (2) pull the skin up and away at a position adjacent the lower cartilage; (3) pull skin up and away at a position adjacent the fatty tissue; and (4) pull the tip of the nose upward and toward the skull. Further, each nasal strip in FIG. 8 has positioning guides that assist a user in consistently placing the nasal strip 805 in its optimum position. The designs in FIG. 8 are similar to the designs discussed with reference to previously discussed designs, so will not be described in detail.

View 801 shows an advanced nasal strip having a vertical beam 808, an upper lateral beam 812 and a lower lateral beam 815. In this design, the upper portion of the vertical beam is generally wider at the top and bottom, and somewhat more narrow just before the upper lateral beam extends. The upper lateral beam 812 is angled downward in relation to the vertical beam portion 808 and has the same height as it extends outward. In this design, the upper lateral beam 812 comprises two generally parallel strips, both approximately the same height. The lower lateral beam 815 shows an advanced nasal strip having a vertical beam 808, an upper lateral beam 812 and a lower lateral beam 815. In this design, the upper portion of the vertical beam is generally the same height as it extends outward. It will be understood that many other dimensions, angles, and geometries may be used consistent with this disclosure.

It will be understood that in alternative designs the upper lateral beam may be made with multiple strips, or that the lower lateral beam may be made with multiple strips. It will be understood that many other dimensions, angles, and geometries may be used consistent with this disclosure.

View 802 shows an advanced nasal strip having a vertical beam 808, an upper lateral beam 812 and a lower lateral beam 815. In this design, the vertical beam is generally the same width for its entire length. The upper lateral beam 812 is angled downward and curves slightly in relation to the vertical beam portion 808. The upper lateral beam 812 extends along the outer edge of the nose, and ends near the fatty tissue. In this way, the upper lateral beam 812 pulls upward and outward both on the upper cartilage and on the fatty tissue. In this design the lower lateral beam 815 is generally triangular shaped and short as compared to the upper lateral beam 812.

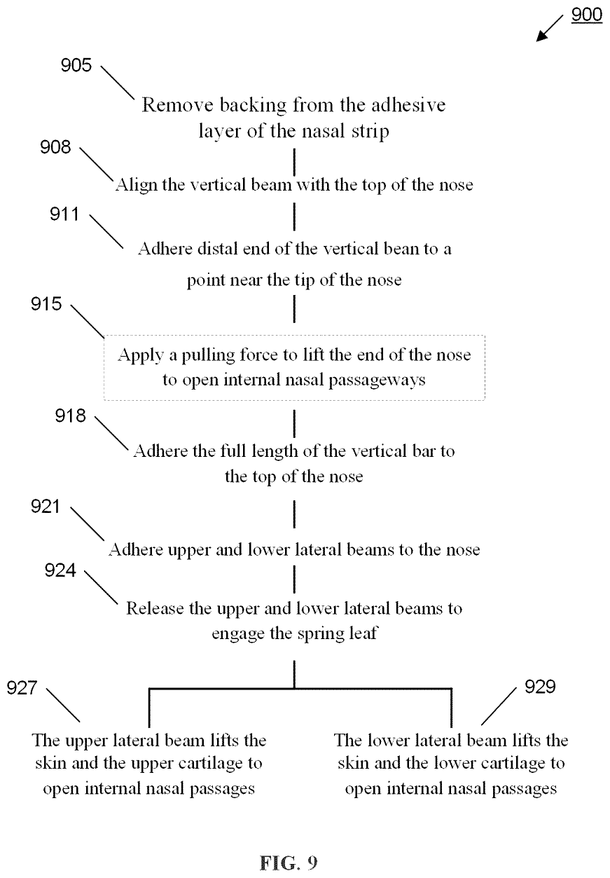

FIG. 9 shows a process 900 for using an advanced nasal strip. First, a user removes the paper backing 905 from the nasal strip to expose the adhesive backing. The user positions 908 the nasal strip so that the vertical beam is aligned with the top of the user's nose. The user then adheres 911 the distal end of the vertical beam to a point near the end of the nose. The user applies a pulling force 915 to pull the end of the nose upward. It will be understood that this pulling force is optional but can result in an increase in air flow through the internal nasal air passageways. The user can adjust this force to balance comfort and improved air flow. When the desired lift is achieved, the user adheres the full length 918 of the vertical beam to the nose. This acts to pull the end of the nose upward, which opens the internal nasal airway. At this point, the nasal strip is properly aligned with the user's nose. The user then presses down the upper and lower lateral beams 921 to adhere them to the sides of the nose. When fully adhered to the skin, the user releases the pressure 924 so that the leaf spring effect in the middle layer of the nasal strip is activated. The leaf spring effect in the upper lateral beam 927 acts to pull the skin of nose and the upper cartilage upward and away, which opens the internal nasal passage, and the lower lateral beam acts 929 to pull the skin of nose and the lower cartilage upward and away, which further opens the internal nasal passage.

While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims. It will also be understood that dimensions and geometries are approximate and are provided only for ease of description.

What is claimed is:

1. A nasal strip for enhancing air flow in a nose of a user by enlarging nasal air passageways, the nose having an internal structure that includes a nasal bone, an upper cartilage, a lower cartilage and fatty tissue, the nasal strip comprising:
an adhesive backing layer for removable attachment to the nose;
a middle leaf spring layer;
a covering layer;
a vertical beam portion sized and constructed to extend from near a distal end of the nose to a point generally between the user's eyes, the vertical beam generally aligning with the nasal bone;
an upper lateral beam extending from the vertical beam portion along both sides of the nose and generally adjacent the upper cartilage;
a lower lateral beam extending from the vertical beam portion along both sides of the nose and generally adjacent the lower cartilage;
wherein when the upper lateral beam is adhered to the nose, a portion of the leaf spring layer acts to pull the upper cartilage up and away to expand internal nasal passageways; and
wherein when the lower lateral beam is adhered to the nose, a portion of the leaf spring layer acts to pull the lower cartilage up and away to expand internal nasal passageways.

2. The nasal strip according to claim 1, wherein the vertical beam portion is constructed to apply a lifting force to the distal end of the nose when the vertical beam portion is adhered to the nose, which acts to lift the end of the nose and expand internal nasal passageways.

3. The nasal strip according to claim 1, wherein the lower lateral beam is further sized and constructed to lift the fatty tissue up and away, which acts to expand internal nasal passageways.

4. The nasal strip according to claim 1, wherein the nasal strip is constructed as a unitary assembly.

5. The nasal strip according to claim 1, wherein the leaf spring layer is constructed from polyester, PET, plastic, metal, or memory metal.

6. The nasal strip according to claim 1, wherein the covering layer is constructed from a fabric, rayon, or paper.

7. The nasal strip according to claim 1, wherein the upper lateral beam or the lower lateral beam is curved with respect to the vertical beam portion.

8. The nasal strip according to claim 1, wherein the upper lateral beam or the lower lateral beam is constructed from a plurality of strips.

9. The nasal strip according to claim 1, wherein the upper lateral beam and the lower lateral beam are connected.

10. The nasal strip according to claim 1, wherein the vertical beam portion has a void.

11. The nasal strip according to claim 1, wherein the upper lateral beam or the lower lateral beam angle downward with respect to the vertical beam portion.

12. The nasal strip according to claim 1, wherein the upper lateral beam or the lower lateral beam angle upward with respect to the vertical beam portion.

13. The nasal strip according to claim 1, wherein the upper lateral beam and the lower lateral beam are generally parallel to each other.

14. A leaf spring for a nasal strip for enhancing air flow in a nose of a user by enlarging nasal air passageways, the nose having an internal structure that includes a nasal bone, an upper cartilage, a lower cartilage and fatty tissue, the leaf spring comprising:
a vertical beam portion sized and constructed to extend from near a distal end of the nose to a point generally between the user's eyes, the vertical beam generally aligning with the nasal bone;
an upper lateral beam extending from the vertical beam portion along both sides of the nose and generally adjacent the upper cartilage;
a lower lateral beam extending from the vertical beam portion along both sides of the nose and generally adjacent the lower cartilage;
wherein when the upper lateral beam is adhered to the nose, a portion of the leaf spring layer acts to pull the upper cartilage up and away to expand internal nasal passageways; and
wherein when the lower lateral beam is adhered to the nose, a portion of the leaf spring layer acts to pull the lower cartilage up and away to expand internal nasal passageways.

15. The leaf spring according to claim 14, wherein the leaf spring is constructed from polyester, PET, plastic, metal, or memory metal.

16. The leaf spring according to claim 14, wherein the upper lateral beam or the lower lateral beam is curved with respect to the vertical beam portion.

17. The leaf spring according to claim 14, wherein the upper lateral beam or the lower lateral beam is constructed from a plurality of strips.

18. The leaf spring according to claim 14, wherein the upper lateral beam and the lower lateral beam are connected.

19. The leaf spring according to claim 14, wherein the vertical beam portion has a void.

20. The leaf spring according to claim 14, wherein the upper lateral beam or the lower lateral beam angle downward with respect to the vertical beam portion.

21. The leaf spring according to claim 14, wherein the upper lateral beam or the lower lateral beam angle upward with respect to the vertical beam portion.

22. The leaf spring according to claim 14, wherein the upper lateral beam and the lower lateral beam are generally parallel to each other.

23. A method of using a nasal strip for enhancing air flow in a nose of a user by enlarging nasal air passageways, the nose having an internal structure that includes a nasal bone, an upper cartilage, a lower cartilage and fatty tissue, the nasal strip comprising:
adhering a vertical beam portion of the nasal strip to a point near the nose's tip;
adhering the vertical beam portion to the nose's top and generally aligning with the nasal bone, which aligns the nasal strip correctly on the nose;
adhering an upper lateral beam to the nose, which is generally aligned with the upper cartilage;
adhering a lower lateral beam to the nose, which is generally aligned with the lower cartilage; and
wherein when fully adhered to the nose, the upper lateral beam lifts the upper cartilage up and away, and the lower lateral beam lifts the lower cartilage up and away, which act to expand internal nasal passageways.

24. The method according to claim 23, further comprising the step of applying a lifting force to the distal end of the nose when the vertical beam portion is being adhered to the nose, which acts to lift the end of the nose and expand internal nasal passageways.

25. The method according to claim 23, further comprising the step of using the lower lateral beam to lift the fatty tissue up and away, which acts to expand internal nasal passageways.
